(19) United States
(12) Patent Application Publication
(10) Pub. No.: US 2023/0340043 A1
(43) Pub. Date: Oct. 26, 2023

(54) THERAPEUTIC PEPTIDE COMPOSITION AND METHODS OF TREATMENT

(71) Applicants: Archana Varadaraj, Camarillo, CA (US); Narendiran Rajasekaran, Camarillo, CA (US)

(72) Inventors: Archana Varadaraj, Camarillo, CA (US); Narendiran Rajasekaran, Camarillo, CA (US)

(21) Appl. No.: 18/139,810

(22) Filed: Apr. 26, 2023

Related U.S. Application Data
(60) Provisional application No. 63/335,130, filed on Apr. 26, 2022.

(51) Int. Cl. C07K 14/47; A61P 11/00; C12N 15/86

(52) U.S. Cl. CPC C07K 14/4703; A61P 11/00; C12N 15/86; A61K 38/00

(57) ABSTRACT

During lung fibrosis, activation of fibroblasts to myofibroblasts, is accompanied by the increased assembly of extracellular matrix proteins fibronectin and collagen. Activated myofibroblasts are key drivers of fibrosis; and increased deposition of the assembled matrix proteins, preserves the myofibroblast state. These changes contribute to enhanced tissue stiffness, collagenous deposition known as fibroblastic foci in lung and therefore, established pulmonary fibrosis. The present invention identifies a novel function of the 40 amino acid SOCS domain peptide (SEQ ID NO:1), in degrading the pathologic fibronectin and collagen matrix associated with fibrosis, markedly reducing the myofibroblast protein α-SMA and reducing collagen deposits in the fibrotic lung, leading to fibrosis reversal. Use of the SOCS domain peptide is a new approach to treat lung fibrosis as it restricts extracellular matrix assembly and reverses myofibroblasts to fibroblasts.

### CROSS-REFERENCE TO RELATED APPLICATIONS

This application claims the priority benefit of U.S. Provisional Application Ser. No. 63/335,130, filed on Apr. 26, 2022, which is incorporated herein by reference.

### STATEMENT REGARDING FEDERAL FUNDING

This invention was made with government support under grant number R15HL154051-01A1 awarded by the National Institutes of Health. The government has certain rights in the invention.

### INCORPORATION-BY-REFERENCE OF SEQUENCE LISTING

The text of the computer readable sequences listing filed herewith, titled “63335130_Sequence_Listing.xml”, created Apr. 26, 2023, having a file size of 3,000 bytes, is hereby incorporated by reference in its entirety.

### FIELD OF THE INVENTION

The present invention is directed to methods and composition for disassembly of the fibronectin and collagen matrix and reducing levels of smooth muscle actin (α-SMA) in fibrotic lung fibroblasts, restricting differentiation of fibroblasts to fibrotic lung fibroblasts and reducing collagen deposition in fibrotic lung, using SOCS domain peptide. In particular, the use for the reversal of fibrotic lung fibroblasts to normal fibroblast cells and treatment of lung fibrosis are considered.

### INTRODUCTION

Idiopathic pulmonary fibrosis (IPF) is a rapidly progressive and irreversible disease with a median survival of 2.5-3.5 years (Flaherty, Thwaite et al. 2003, Perez, Rogers et al. 2003, Monaghan, Wells et al. 2004). IPF is the most common of interstitial lung disease overall, with patients experiencing progressive breathlessness, eventual respiratory failure and death. With 34,000 new cases diagnosed each year, IPF poses a public health problem in the United States (Raghu, Weycker et al. 2006).

The lungs in an IPF patient present a characteristic patchy fibrosis along the inferior portions of the lobes, with areas of established fibrosis, fibroblastic proliferation and collagenous deposition known as fibroblastic foci (Jones, Fabre et al. 2016). Recurrent lung injury and the failure to resolve the wound-healing process leads to excessive accumulation of extracellular matrix (ECM) proteins, leading to loss in lung function.

Recent clinical trials using drugs that target receptor tyrosine kinase and TGFβ, Nintedanib and Pirfenidone respectively, show increased disease-free survival by slowing down the differentiation of fibroblasts to myofibroblasts (Noble, Albera et al. 2011, King, Bradford et al. 2014, Richeldi, du Bois et al. 2014, Fala 2015). Neither drug restores the fibrotic lung to normalcy. Approaches that reverse the disease is an unmet healthcare need.

IPF progresses in two discrete phases, an initial inflammatory phase with elevated levels of pro-inflammatory cytokines followed by the fibrotic phase characterized by increased expression of extracellular matrix proteins including collagen and fibronectin. While historically, IPF was considered to be caused by pro-inflammatory signaling, current research has defined the cause of IPF to aberrant wound closure in alveolar epithelial cells (King, Bradford et al. 2014). Treatment by sustained inhibition of inflammatory pathways is counterproductive to disease resolution due to altered immunomodulatory effects that hinder the wound healing process that is required for fibrosis reversal (Linke, Goren et al. 2010). Anti-inflammatory inhibitors have also shown little success in resolving the wound healing process (Raghu, Brown et al. 2008, Linke, Goren et al. 2010, Raghu, Martinez et al. 2015).

(Nakashima, Yokoyama et al. 2008) describes a method of ameliorating disease severity in a bleomycin-induced IPF mouse model, with adenoviral SOCS1 (Suppressor Of Cytokine Signaling) gene transfer to the lung, 48 hr prior to fibrosis disease induction. In this study the anti-inflammatory function of SOCS1 is exploited, prior to disease initiation, to retard the progression of the disease. The SOCS1 protein is not used to target the fibrotic phase of the disease and does not serve as a therapeutic intervention modality.

Within the SOCS family of proteins SOCS 1-7 and cytokine inducible SH2-containing protein (CISH), the SOCS domain (SEQ ID NO:1) is a 40 amino acid conserved region not demonstrated to exhibit anti-inflammatory or immunosuppressive function (Piganis, De Weerd et al. 2011). Studies by the present inventor and colleagues showed that viral proteins comprising the SOCS domain forms a Cullin-Ring ligase (CRL) complex targeting the Von-Hippel Lindau (VHL) tumor suppressor protein for ubiquitin-mediated degradation (Pozzebon, Varadaraj et al. 2013).

VHL is upregulated in IPF patients and can independently contribute to the fibrotic state when overexpressed (Zhou, Pardo et al. 2011). Studies in renal cancer cells showed that VHL directly interacts with the ECM protein fibronectin to initiate the assembly or polymerization of the fibronectin protein by a process called fibrillogenesis (Ohh, Yauch et al. 1998, Hoffman, Ohh et al. 2001). The polymerization of fibronectin is required and precedes collagen assembly and deposition (McDonald, Kelley et al. 1982, Veiling, Risteli et al. 2002, To and Midwood 2011). Incidentally, the pathological secretion and assembly of fibronectin and collagen (Muro, Moretti et al. 2008, Altrock, Sens et al. 2015) accompanies the differentiation of fibroblasts to myofibroblasts, ECM deposition and increased matrix stiffness during fibrosis. The increased stiffness of the ECM converts mechanical stimuli to fibrogenic stimuli by activating profibrotic cytokines such as TGFβ from its latent form. TGFβ further increases matrix assembly, cell contractility and maintenance of fibrotic fibroblasts.

There is a need therefore to identify novel molecular tools that reduce or retard matrix assembly in fibroblasts in order to arrest and reverse fibrosis. It is against this background that the present inventors have identified a peptide for the disassembly of the matrix and reversal of fibrosis.

### SUMMARY OF THE INVENTION

We discovered that the 40 amino acid SOCS domain peptide VRPLQELCRQRIVAAVGRENLARIPLNPVLRDYLSSFPFQI (SEQ ID NO:1) reversed differentiation of fibroblasts to myofibroblasts, significantly reduced assembly of fibronectin and collagen matrix proteins and restricted the differentiation of fibroblast to myofibroblast in in vitro models of fibrosis. The present inventors also discovered that the SOCS domain peptide (SEQ ID NO:1) reduced collagen accumulation in bleomycin-induced IPF in vivo and reduced subsequent lung damage.

The present invention relates to the novel function of the SOCS domain peptide in degrading the fibronectin and collagen matrix in fibrotic fibroblasts.

In a first aspect, the invention provides a composition comprising a 40 amino acid peptide conserved in the SOCS family of proteins and designated the SOCS domain peptide with the following sequence VRPLQELCRQRIVAAVGRENLARIPLNPVLRDYLSSFPFQI (SEQ ID NO:1). The amino acid SOCS domain peptide when mutated at two key residues, VRPPQELFRQRIVAAVGRENLARIPLNPVLRDYLSSFPFQI (SOCS domain mutant, SEQ ID NO:2) abrogates its function to degrade the fibronectin or collagen matrix.

The present invention also provides a method for decreasing the fibronectin and collagen assemblies in fibrotic fibroblasts. Unlike normal fibroblasts in the lung, fibrotic fibroblasts, interchangeably referred to as myofibroblasts, secrete and assemble increased amounts of extracellular matrix proteins fibronectin and collagen. The fibrotic matrix assemblies, exacerbates disease state and compromises organ function. Targeting the matrix for disassembly and reversing the disease is a promising new approach to restore lung function.

We believe that the degradation of VHL by the SOCS domain peptide is probably one of the mechanisms but not the sole mechanism by which the fibronectin matrix is disrupted.

Importantly, the SOCS domain peptide (SEQ ID NO:1) significantly reduced levels of the myofibroblast marker protein α-SMA, in fibroblasts differentiated to myofibroblasts, indicating a reversal of the myofibroblast cell state in in vitro fibrosis. The SOCS domain mutant was unable to reduce α-SMA, indicating that the two mutated amino acid residues present in the SOCS domain mutant (SEQ ID NO:2) is critical to the function of the SOCS domain in regulating α-SMA and its consequent reactions.

We have demonstrated that introducing the SOCS domain peptide to fibroblast cells, restricts differentiation towards a myofibroblast phenotype, despite treatment of these cells with the profibrotic cytokine TGFβ. Therefore, SOCS domain peptide mediated mitigation of IPF represents a novel prophylactic approach in treatment of fibrosis.

In a bleomycin mouse model of IPF, the SOCS domain peptide, introduced after fibrosis initiation, significantly reduced the collagen content in the fibrotic lung. Collagen content impacts the normal functioning of the lung and plays an active role in defining the extent of organ failure. Collagen accumulation exceeds its degradation during fibrosis. Thus new strategies that effectively target the matrix are required to advance effective treatment modalities.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: SOCS domain degrades VHL protein in myofibroblasts. (FIG. 1A) Lung fibroblasts were untreated or differentiated to myofibroblasts with 5 ng/mL TGFβ for 48 h. Myofibroblasts transduced with Ad-Empty 100 MOI, Ad-Domain 600 MOI or Ad-Mutant 600 MOI for ˜36 h are indicated. Cells were lysed in SDS lysis buffer and immunoblotted for GFP (FIG. 1A), VHL (FIG. 1B) and HIF-1α (FIG. 1C). β-Actin and β-Tubulin are loading controls. Bar graphs in FIG. 1C and FIG. 1E depict arbitrary densitometric units quantified from immunoblots for VHL and HIF-1α respectively and are represented as averages of three independent trials. Statistical significance and P-values (*P<0.05, **P<0.01) were determined using Student's t-test.

FIGS. 2A-2D: SOCS domain fragments FN matrix and reduces levels of SMA protein in myofibroblasts. (FIG. 1A) IMR90 lung fibroblasts were untreated or differentiated to myofibroblasts with 5 ng/mL TGFβ for 48 h. Myofibroblasts transduced with Ad-Empty (100 MOI), Ad-Domain (600 MOI) or Ad-Mutant (600 MOI) for ˜36 h are indicated. The cells were immunostained for fibronectin (FN, red, collagen (COL, red) or α-SMA (red) and nuclear stain DAPI (blue). Myofibroblasts transduced with Ad-Empty (100 MOI), Ad-Domain (600 MOI) or Ad-Mutant (600 MOI) are shown in green (GFP reporter). Scale bar=10 μM. (FIG. 2B) Bar graphs depict the percentage of fibronectin fibril-containing cells, in each condition as shown on the x-axis of the graph. Only GFP reporter transduced cells in green were included in the analysis. n=100 cells/trial; bar graphs represent averages of three independent trials. (FIG. 2C) depicts bar graphs of percentage cells with collagen fibrils, quantified and analyzed exactly as in FIG. 2B. (FIG. 2D) Bar graph depicts arbitrary units of SMA intensities acquired from GFP transduced cells, in each condition as shown on the x-axis of the graph. Graph is representative of average intensities from three independent experiments. Statistical significance and P-values (**P<0.01, ***P<0.001) were determined using Student's t-test.

FIGS. 3A-C. SOCS domain restricts differentiation of fibroblasts to myofibroblasts. (FIG. 3A) IMR90 lung fibroblasts were untreated or transduced with Ad-Empty (100 MOI), Ad-Domain (600 MOI) or Ad-Mutant (600 MOI) for ˜36 h. Untreated fibroblasts and the transduced fibroblasts were differentiated with ng/mL TGFβ for 48 h. Fibroblasts, myofibroblasts and transduced myofibroblasts were evaluated for FN (red) or SMA (red) and nuclear stain DAPI (blue) by immunostaining. Myofibroblasts transduced with Ad-Empty (100 MOI), Ad-Domain (600 MOI) or Ad-Mutant (600 MOI) are shown in green (GFP reporter). Scale bar=10 μM.

(FIG. 3B) Transduced cells as in (FIG. 3A) were quantified for percent cells containing fibronectin fibrils and represented as bar graphs. n=100 cells/trial; bar graphs represent averages of three independent trials. (FIG. 3C) Bar graph depicts arbitrary units of α-SMA intensities acquired from GFP transduced cells, in each condition as shown on the x-axis of the graph. n=100 cells/trial; bar graphs represent averages of three independent trials.

FIG. 4A-C. Intranasal delivery of Adenoviral-GFP is localized to lung tissue. (FIG. 4A) C57BL/6 mice were intranasally administered vehicle (phosphate buffered saline) and adenoviral-GFP on Day 0 at 10⁸ PFU and 10⁹ PFU as depicted. Lung tissue was collected on Day 5 and Formalin Fixed paraffin embedded (FFPE) lung tissue was subjected to immunohistochemistry (IHC) staining using GFP-DAB antibody. Increased transduction of Ad-GFP at 10⁹ PFU was observed compared to transduction at 10⁸ PFU 5 days following intranasal delivery of Ad-GFP and vehicle control (n=3). Magnification=20×. Scale bar=10 μm. (FIG. 4B) FFPE lung tissue sections analyzed for GFP expression. Top row depicts GFP-DAB staining and bottom row shows nuclei (blue). (FIG. 4C) Positive stain intensity from the bottom row in FIG. 4B, assigned using the scores: 1+ yellow, 2+ orange and 3+ red was used to quantify extent and intensity of GFP represented using the histoscore (H-score). Bar graph represents mean±SD, n=3. Statistical significance between vehicle (phosphate buffered saline) versus transduced and P-values (*P<0.05, ***P<0.001) are depicted.

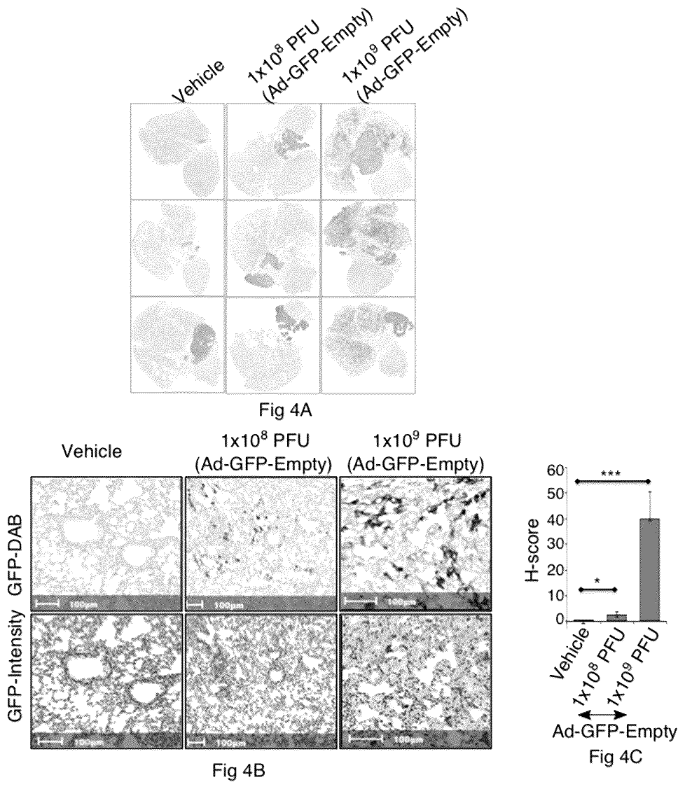

FIG. 5A. SOCS domain significantly reduces collagen deposition in fibrotic lung. C57BL/6 mice were administered bleomycin by oropharyngeal aspiration on DO and intranasally administered adenovirus on D9. Lung lobes collected on D21 was quantified for collagen deposits (hydroxyproline assay). Disease vs SOCS domain *p<0.05. n=6 mice per group.

### DETAILED DESCRIPTION OF THE INVENTION

Provided herein is a 40 amino acid SOCS domain polypeptide composition and method of delivery for the treatment of lung fibrosis.

Current understanding in the field of IPF, demonstrates that lung fibrosis is accompanied and driven by the deposition and assembly of extracellular matrix proteins fibronectin and collagen. The present inventors confirmed that normal lung fibroblast cells differentiated to their fibrotic counterpart, myofibroblasts, markedly enhance matrix formation of fibronectin and collagen. Matrix formation is readily detectable by fluorescence microscopy, and labeling of specific proteins using fluorophore-conjugated antibodies revealing fibril-like structures greater than 3 μm in length. While the persistence of the matrix contributes to the pathogenesis of IPF, a method to target the matrix and restore the myofibroblast cells towards the fibroblast state has been unclear.

IPF patient lungs have demonstrated elevated VHL levels. The VHL protein is central to the process of matrix formation of fibronectin. The present invention reduces both VHL levels and matrix formation thereby effectively targeting both components that drive fibrosis.

Unlike normal lung fibroblasts, fibroblasts obtained from lungs of patients with IPF or from mice with bleomycin-induced IPF, an accepted model of human IPF, collagen content is vastly increased. As demonstrated in the present invention, the SOCS domain significantly reduces collagen accumulation in the IPF lung.

We have demonstrated the protective function of the SOCS domain in restricting the conversion of normal lung fibroblasts towards their fibrotic counterpart, myofibroblasts, if the SOCS domain is overexpressed in these cells. Here, the SOCS domain prevents assembly of the fibronectin matrix and maintains the cells in the normal fibroblast state.

### Definitions

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein, the term ‘treatment’ or ‘therapeutic’ means an approach to obtaining a beneficial or intended clinical result, which may include alleviation of symptoms, reduction in disease severity, inhibiting the underlying cause of the disease, stabilizing diseases in a non-advanced state, preventing disease progression, delaying the progress of disease and/or improvement or alleviation of disease conditions.

The term “fibroblast” as used herein refers to a connective tissue cell that makes and secretes collagen protein. Fibroblasts, the most common cell type found in connective tissues, play as important role in healing wounds. In certain situations, epithelial cells can give rise to fibroblasts, a process called epithelial-mesenchymal transition.

The term “myofibroblasts” used interchangeably with “fibrotic fibroblasts” and used herein, refers to the activated fibroblasts in the wound areas that have some characteristics of smooth muscle, express α-SMA, exhibit contractile properties and produce excess fibronectin and collagen.

The term “fibronectin assembly” or fibrillogenesis used herein refers to the assembly of fibronectin protein dimers into fibronectin polymers that localize at the cell surface.

The term “matrix assembly” refers to the assembly or polymerization of matrix proteins contributing to matrix stiffness, cell stiffness and mechanical properties of the cell.

In embodiments where compositions are provided comprising the SOCS domain, the SOCS domain polypeptide may be synthetic (e.g., designed by man) or naturally occurring sequence.

As referred to herein, “polypeptide” and “peptide” may be used interchangeably.

Suitably, the test peptide or polypeptide may be labeled with an antibody, a tag, a reporter or may be a fluorescent protein fusion.

In another embodiment of any aspect of the invention, the test peptide or polypeptide is functionally linked to a reporter protein, and wherein detecting the amount of test peptide present in the cell comprises detecting the amount of reporter protein present in the cell.

The candidate peptide in accordance with any aspect of the invention may be provided by expression of one or more nucleic acid molecules encoding the candidate peptide. Suitably, the nucleic acid sequences encoding the candidate peptide may be encompassed in different plasmids/vectors such that transfection/transduction of a eukaryotic cell with each plasmid/vector will result in each component (peptide/polypeptide) being produced by the cell, when it is cultured in suitable conditions.

In embodiments in which the SOCS domain polypeptide is provided, the SOCS domain polypeptide retains one or more structural and/or functional features of the full-length protein such as VHL degradation, HIF-1α upregulation, matrix degradation.

In embodiments in which the SOCS domain polypeptide is provided, the SOCS domain polypeptide does not retain one or more structural and/or functional features of the full-length protein such as capability to attenuate STAT phosphorylation, inhibit cytokine induction of STAT, suppress inflammatory gene expression, etc.

As used herein, the term “SOCS domain mutant” refers to a variant of the SOCS domain polypeptide having a distinct amino acid sequence from the most common variant occurring in nature, referred to as the “wild-type” sequence. A mutant polypeptide may be a naturally-occurring protein that is not the most common sequence in nature (or a subsequence thereof), or may be a polypeptide that is not a naturally-occurring sequence (or a subsequence thereof). For example, a “SOCS domain mutant” may be a naturally-occurring, non-wild-type SOCS domain, or may be a synthetic SOCS domain that does not occur in nature.

As used herein, the term “pharmaceutical composition” refers to the combination of an active agent (e.g., SOCS domain peptide) with a carrier, inert or active, making the composition especially suitable for diagnostic or therapeutic use in vitro, in vivo or ex vivo.

The term “pharmaceutically acceptable”, as used herein, refers to compositions that do not substantially produce adverse reactions, e.g., toxic, allergic, or immunological reactions, when administered to a subject.

The peptide composition may contain suitable excipients such as lysozyme, BSA, cellular retinoic acid-binding protein, yeast hexokinase, recombinant human insulin, β-lactoglobulin, invertase (EC 3.2.1.26), apoflavodoxin, amyloidogenic light chain protein, ribonuclease-A, recombinant hemoglobin, albutropin, hGH, trehalose, sucrose, lactose, mannitol, sorbitol, dextran, PVP, starch derivatives, polysorbates 20 and 80, poloxamer, PEG, polyethylene glycol-b-poly L-histidine. ascorbic acid, ecotoine, glutathione, monothioglycerol, morin, polyethylenimine, propyl gallate, vitamin E, citric acid, EDTA, hexaphosphate, thioglycolic acid, phosphate, bicarbonate, sulphate, nitrate, acetate, chloride, pyruvate, Mg(OH)₂, ZnCO₃, alanine, arginine, aspartic acid, glycine, histidine, lysine, proline, glucose, potassium phosphate, sodium sulphate, hexaphosphate, phenol, zinc, cyclodextrin, glycerol.

### Example I

### SOCS Domain Decreases VHL Levels in Lung Myofibroblasts

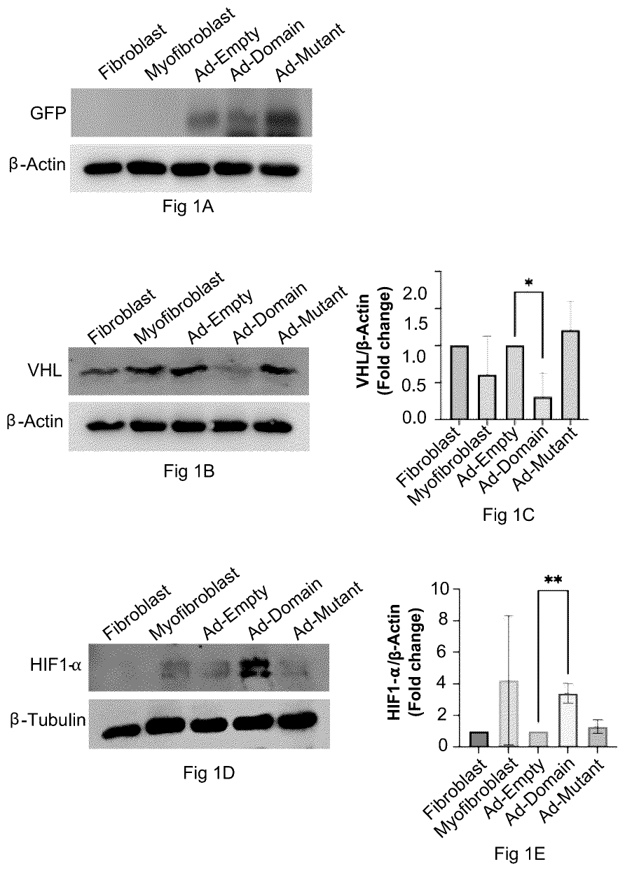

To test whether the SOCS domain degrades VHL in myofibroblasts, IMR90 lung fibroblasts were differentiated to myofibroblasts with TGFβ treatment for 48 h and cells transduced with adenovirus expressing the SOCS domain, SOCS domain mutant or empty viral vector. Viral transduction of myofibroblast cells are evident from GFP reporter expression. As depicted in the immunoblot, GFP expression was confirmed (FIG. 1A) and VHL depletion observed specifically in cells transduced with the SOCS domain (FIG. 1B and FIG. 1C). Bona fide VHL depletion preserves HIF-1α protein levels, which is confirmed by increase in HIF-1α protein levels in the SOCS domain transduced cells (FIG. 1D and FIG. 1E). Together, these findings confirm that the SOCS domain (unlike the mutant) decreases VHL protein levels in myofibroblast cells.

### Example II

### SOCS Domain Promotes Matrix Disassembly and Decreases αSMA in Myofibroblasts

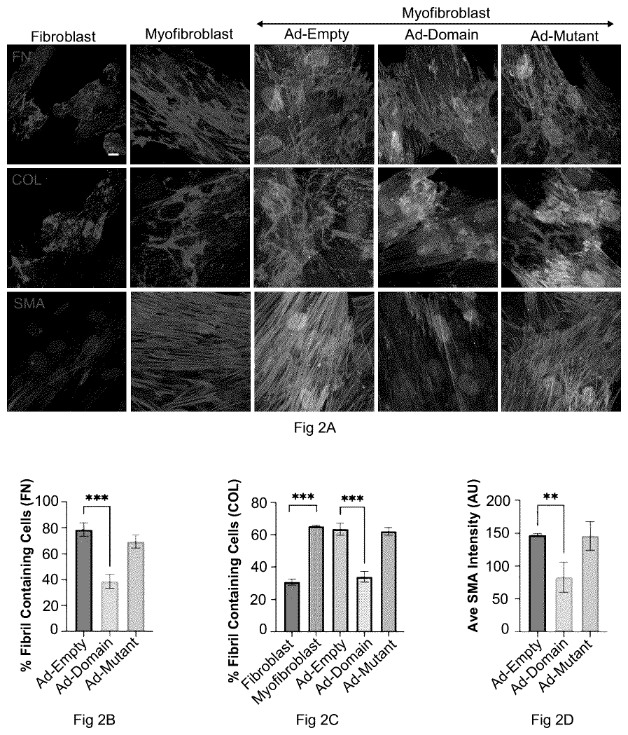

Prior studies have established that excess matrix deposition and assembly, preserves a fibrotic state (King, Pardo et al. 2011). As confirmed by immunocytochemistry, differentiation of fibroblasts to myofibroblasts using TGFβ increased fibril formation of fibronectin and collagen (FIG. 2A). Levels of α-SMA, a marker for contractile myofibroblasts was also increased upon TGFβ treatment, confirming the fibrotic cell state. To evaluate whether the SOCS domain abrogates matrix formation in myofibroblasts, myofibroblasts were transduced with the SOCS domain, SOCS domain mutant or the empty viral vector (same as in FIG. 1). A significant decrease in the assembly of ECM proteins fibronectin and collagen were observed in SOCS domain transduced cells. In these conditions, a significant decrease in levels of α-SMA was evident. However, no significant decrease in fibril assembly of matrix proteins or α-SMA levels was observed in myofibroblasts transduced with the SOCS domain mutant. Quantification of the transduced cells (as determined from GFP reporter expression) confirmed the significant decrease in matrix fibrils and reduced levels of α-SMA, in cells expressing the SOCS domain (FIG. 2B-FIG. 2D).

### Example III

### SOCS Domain Retards Fibroblast Differentiation

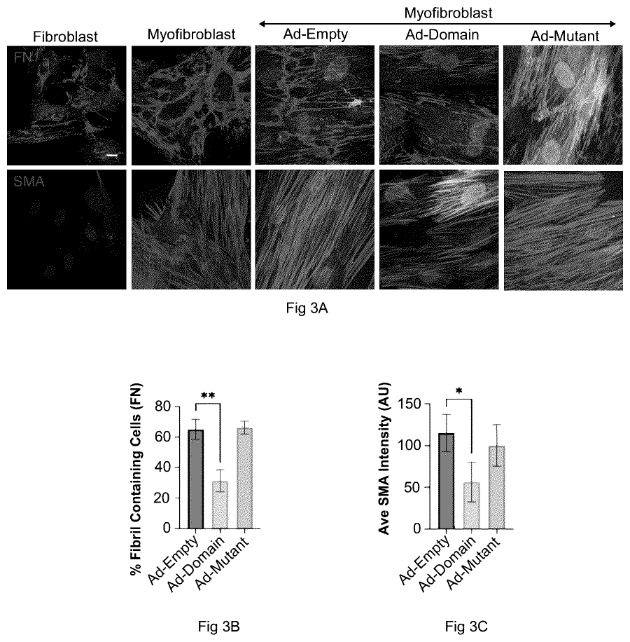

Having shown that the SOCS domain significantly restores fibroblasts after differentiation by TGFβ, the goal of the next experiment was to test whether the SOCS domain retards fibroblast differentiation. Here, fibroblast cells were transduced with adenovirus expressing the SOCS domain, mutant or empty vector, prior to differentiation with TGFβ. Untransduced cells exhibited a noticeable increase in fibronectin matrix deposition and α-SMA levels upon differentiation. However, fibroblast cells transduced with the SOCS domain failed to upregulate matrix formation or α-SMA levels when differentiated with TGFβ (FIG. 3A-C). These data strongly demonstrate that SOCS domain expression in fibroblast cells retards fibroblast differentiation.

### Example IV

### Intranasal Delivery of Adenoviral Vectors Localized to the Lung

Localization and expression of adenovirus-mediated transduction to mouse lungs was confirmed by intranasal delivery of empty adenovirus vector expressing the GFP reporter. As shown in FIG. A-C, viral titers of 1×10⁸ PFU and 1×10⁹ PFU were delivered on Day 0 and mouse lung tissue was evaluated on Day 5 for GFP-DAB levels by immunohistochemistry. Substantial GFP expression was detected at 1×10⁹ PFU compared to the negligible staining at 1×10⁸ PFU. GFP expression was restricted to the lung tissue and we observed no expression in the diaphragmatic smooth muscle in close proximity to the lungs and no expression was noted in the lymphoid tissue found within and near the lungs. Quantification of the GFP intensities (H-score) confirmed significant increase in GFP levels at the higher viral titer. This data confirmed localization and optimum viral titer for transduction to the mouse lung.

### Example V

### SOCS Domain Reduces Fibrotic Changes in the In Vivo Model of Bleomycin-Induced IPF

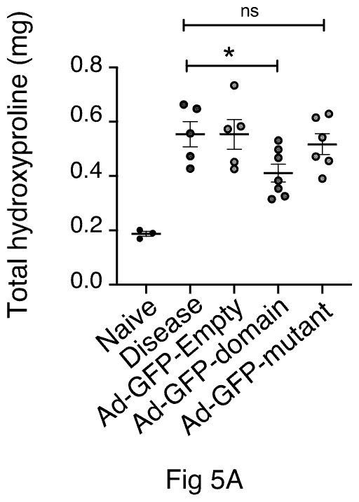

Here, IPF was established in a bleomycin (BLM) mouse model of lung fibrosis by delivery of bleomycin 3.07 units/kg on Day 0. On Day 9, during the fibrotic phase of the disease, vehicle (saline) and adenovirus (SOCS domain, SOCS domain mutant and Empty vector) was administered intranasally at a viral titer of 1×10⁹ PFU. On Day 21 after initiation of bleomycin injury, mouse lung tissue was collected, lysed and analyzed for collagen content using the hydroxyproline assay (FIG. 5A). Although collagen content was elevated in the disease lung, lung tissue transduced with the Empty vector and SOCS domain mutant, a significant decrease in the hydroxyproline content was noted only in mouse lung transduced with the SOCS domain. The data demonstrate the specific and translatable function of the SOCS domain in degrading the lung matrix that would otherwise sustain and preserve fibrosis.

### Example VI

### Materials and Methods

Cell Lines and Culture Conditions

Human lung fibroblasts, IMR90, were obtained from the American Type Culture Collection (ATCC, #CCL-186) and cultured in EMEM media (ATCC, #30-2003) supplemented with 10% FBS (HyClone, #SH30109.03). Cells were maintained in a 37° C. humidified incubator buffered with 5% CO2. All experiments were performed at 70-80% cell densities.

Fibroblast Differentiation Conditions

IMR90 fibroblasts were differentiated to myofibroblasts with 5 ng/mL Recombinant Human Transforming Growth Factor-β1 (TGF-β1) (Invitrogen, #PHG9204) for 48 h in serum free media. TGF-β1 was reconstituted and stored according to manufacturer's recommendations.

Antibodies and Reagents

Antibodies for immunoblotting and immunocytochemistry: GFP (Abcam, #ab183734), VHL (Cell Signaling, #68547S), HIF1-α (Cell Signaling Technology, #14179S), β Actin (Invitrogen, #PA5-59497), β Tubulin (Invitrogen, #MA5-16308), Fibronectin (Santa Cruz, #sc-59826), Collagen (Abcam, #ab34710), Smooth Muscle Actin (Abcam, #ab7817), Donkey Anti-Mouse Alexa Flour 568 (Invitrogen, #A10037), Donkey Anti-Rabbit Alexa Flour 568 (Invitrogen #A10042).

Adenovirus Transduction

Adenoviral vectors Ad-GFP-Empty, Ad-GFP-SOCS domain, Ad-GFP-SOCS domain mutant were purchased from Vector Biolabs at a viral titer of 4.5×10¹⁰ PFU/mL, 1.2×10¹¹ PFU/mL, 1.1×10¹² PFU/mL respectively. We obtained ˜80% transduction for Ad-GFP at 100 MOI, Ad-GFP-SOCS domain at 600 MOI, and Ad-GFP-SOCS domain mutant at 600 MOI for our in vitro experiments.

Protein Extraction and Immunoblotting

IMR90 cells were seeded on 6 cm plates at a density of 163,000 cells. Protein extraction was performed at 4° C. using cold SDS lysis buffer containing protease and phosphatase inhibitors (1 mM DTT, 1 mM EDTA, 1 μg/mL Leupeptin, 100 μg/mL PMSF, and 1 mM Sodium Orthovanadate). Proteins were separated by SDS-Poly acrylamide gel electrophoresis and immunoblotted for specific proteins of interest. β Actin and β Tubulin were used as loading controls. Quantification of immunoblots was performed using the Li-Cor Image Studio Software version 5.2. Pixel intensities of each protein were normalized to the loading control. Three independent experiments were averaged and fold differences between fibroblasts and myofibroblasts or myofibroblasts transduced with Ad-GFP and myofibroblasts transduced with adenoviruses Ad-GFP-SOCS domain or Ad-GFP-SOCS domain mutant were plotted and shown as bar graphs. P values were determined using Student's t-test.

Immunocytochemistry and Imaging

For immunocytochemistry of Fibronectin, SMA, or collagen, IMR90 cells were seeded on sterile coverslips in a 6-well plate at a density of 80,000 cells per well. The coverslips were fixed with 4% paraformaldehyde and permeabilized in Triton X-100 for 1 min on ice. The cells were then blocked with 5% BSA-lx PBS. After blocking, FN antibody (1:70), SMA (1:1000) or COL (1:100) was added for 1 h followed by 1 h incubation with a secondary antibody. After secondary incubation, the cells were washed repeatedly with 0.2% Tween in 1×PBS. After washes, the cells were stained with the DNA stain DAPI (4,6 diamidino-2-phenylindole dihydrochloride) (Roche, #1023627001) and mounded with Prolong gold anti-fade mound media (Invitrogen, #S36936) on glass slides. Images and z-stacks (1 μM z-slice) were acquired using a Leica TCS SPEII confocal microscope at consistent acquisition parameters for each experiment.

Fibril Count and Quantification

Fibronectin and collagen fibril counts were determined from random fields of view within each sample and average percentage of fibril containing cells included in the bar graphs. A fibronectin or collagen track of ˜3 μM in length was considered a fibril. A total of at least 100 cells were counted for each condition. Data in figures that include fibril counts are averages of 3 independent experiments. Student's t-test was used to calculate P values.

SMA Intensities and Quantification

Bar graphs that depict SMA intensities were constructed with the intensity plugin available in Image J. Cell containing random fields of view within each sample were used to quantify the arbitrary SMA intensity values. SMA intensities included in the drawings represent averages of 3 independent experiments. Student's t-test was used to calculate P values.

Animal Models

All protocols concerning animal use were approved by the Institutional Animal Care and Use Committees at the Northern Arizona University and conducted in strict accordance with the National Institutes of Health Guide for the Care and Use of Laboratory Animals. Studies were conducted with 10-12 week old C57BI/6 mice. Mice were housed in a temperature and humidity-controlled pathogen-free facility with a 12:12 hour light:dark cycle. Animals were housed within a limited access rodent facility and kept in groups of a maximum of 4 mice per cage. Mice were housed in polypropylene cages with solid bottoms and wood shavings or corn cobb as bedding material. At study termination, mice were euthanized via anesthesia overdose and exsanguination.

Bleomycin Lung Fibrosis Model and Viral Transduction

IPF was established in C57BI/6 mice (10-12 weeks) by oral aspiration (OA) of bleomycin (3.07 units/kg) on Day 0. Adenovirus (Ad-GFP-Empty, SOCS domain and SOCS domain mutant) was administered intranasally (IN) on Day 9. Animals were euthanized on Day 21 (5 animals in each group).

Collagen content (hydroxyproline assay) in the lung was determined from lung lysates collected on Day 21 of the study.

Immunohistochemistry and GFP-DAB Staining and Quantification

To determine optimum adenoviral titer for transduction, C57BL/6 mice were administered adenovirus 10⁸ PFU and 10⁹ PFU or vehicle control (saline) on Day 0. On Day 3 following euthanasia, the lungs were inflated and fixed in formalin 10%. Tissues were processed in paraffin, sectioned and stained using GFP-DAB for assessment of transduction and H&E. Images were imported in HALO 3.0, regions of interest (lung tissue) annotated, and a quantitative image analysis algorithm performed. The algorithm (Cytonuclear V1.6) was used to segment nuclei based on hematoxylin staining, classify cells as either positive or negative for DAB staining and stratify positively stained cells as being 1+, 2+ or 3+ staining intensity (1+ being weak, 2+ being moderate, 3+ being strong staining intensity). To assign the H-score, a classification system was used to assess the total number of positive cells as well as the intensity of staining of each positive cell (on a scale of 0-3). The formula is calculated as [0*(ratio of negative cells)+1*(ratio of 1+ cells)+2*(ratio of 2+ cells)+3*(ratio of 3+ cells)].

Statistical Analysis

Immunoblots and immunocytochemistry experiments were performed as 3 independent trials and represented as averages ±SEM. Statistical significance for fibrils counts, SMA intensities and immunoblot intensities were calculated using Student's t-test. Statistical significance (P-value) was determined using GraphPad Prism 8.
Altrock, E., C. Sens, C. Wuerfel, M. Vasel, N. Kawelke, S. Dooley, J. Sottile and I. A. Nakchbandi (2015). “Inhibition of fibronectin deposition improves experimental liver fibrosis.” J. Hepatol 62(3): 625-633.
Fala, L. (2015). “Ofev (Nintedanib): First Tyrosine Kinase Inhibitor Approved for the Treatment of Patients with Idiopathic Pulmonary Fibrosis.” Am Health Drug Benefits 8(Spec Feature): 101-104.
Flaherty, K. R., E. L. Thwaite, E. A. Kazerooni, B. H. Gross, G. B. Toews, T. V. Colby, W. D. Travis, J. A. Mumford, S. Murray, A. Flint, J. P. Lynch, 3rd and F. J. Martinez (2003). “Radiological versus histological diagnosis in UIP and NSIP: survival implications.” Thorax 58(2): 143-148.
Hoffman, M. A., M. Ohh, H. Yang, J. M. Klco, M. Ivan and W. G. Kaelin, Jr. (2001). “von Hippel-Lindau protein mutants linked to type 2C VHL disease preserve the ability to downregulate HIF.” Hum Mol Genet 10(10): 1019-1027.
Jones, M. G., A. Fabre, P. Schneider, F. Cinetto, G. Sgalla, M. Mavrogordato, S. Jogai, A. Alzetani, B. G. Marshall, K. M. O'Reilly, J. A. Warner, P. M. Lackie, D. E. Davies, D. M. Hansell, A. G. Nicholson, I. Sinclair, K. K. Brown and L. Richeldi (2016). “Three-dimensional characterization of fibroblast foci in idiopathic pulmonary fibrosis.” JCI Insight 1(5).
King, T. E., Jr., W. Z. Bradford, S. Castro-Bernardini, E. A. Fagan, I. Glaspole, M. K. Glassberg, E. Gorina, P. M. Hopkins, D. Kardatzke, L. Lancaster, D. J. Lederer, S. D. Nathan, C. A. Pereira, S. A. Sahn, R. Sussman, J. J. Swigris, P. W. Noble and A. S. Group (2014). “A phase 3 trial of pirfenidone in patients with idiopathic pulmonary fibrosis.” N Engl J Med 370(22): 2083-2092.
King, T. E., Jr., A. Pardo and M. Selman (2011). “Idiopathic pulmonary fibrosis.” Lancet 378(9807): 1949-1961.
Linke, A., I. Goren, M. R. Bosl, J. Pfeilschifter and S. Frank (2010). “Epithelial overexpression of SOCS-3 in transgenic mice exacerbates wound inflammation in the presence of elevated TGF-beta1.” J Invest Dermatol 130(3): 866-875.
McDonald, J. A., D. G. Kelley and T. J. Broekelmann (1982). “Role of fibronectin in collagen deposition: Fab′ to the gelatin-binding domain of fibronectin inhibits both fibronectin and collagen organization in fibroblast extracellular matrix.” J Cell Biol 92(2): 485-492.
Monaghan, H., A. U. Wells, T. V. Colby, R. M. du Bois, D. M. Hansell and A. G. Nicholson (2004). “Prognostic implications of histologic patterns in multiple surgical lung biopsies from patients with idiopathic interstitial pneumonias.” Chest 125(2): 522-526.
Muro, A. F., F. A. Moretti, B. B. Moore, M. Yan, R. G. Atrasz, C. A. Wilke, K. R. Flaherty, F. J. Martinez, J. L. Tsui, D. Sheppard, F. E. Baralle, G. B. Toews and E. S. White (2008). “An essential role for fibronectin extra type III domain A in pulmonary fibrosis.” Am J Respir Crit Care Med 177(6): 638-645.
Nakashima, T., A. Yokoyama, Y. Onari, H. Shoda, Y. Haruta, N. Hattori, T. Naka and N. Kohno (2008). “Suppressor of cytokine signaling 1 inhibits pulmonary inflammation and fibrosis.” J Allergy Clin Immunol 121(5): 1269-1276.
Noble, P. W., C. Albera, W. Z. Bradford, U. Costabel, M. K. Glassberg, D. Kardatzke, T. E. King, Jr., L. Lancaster, S. A. Sahn, J. Szwarcberg, D. Valeyre, R. M. du Bois and C. S. Group (2011). “Pirfenidone in patients with idiopathic pulmonary fibrosis (CAPACITY): two randomised trials.” Lancet 377(9779): 1760-1769.
Ohh, M., R. L. Yauch, K. M. Lonergan, J. M. Whaley, A. O. Stemmer-Rachamimov, D. N. Louis, B. J. Gavin, N. Kley, W. G. Kaelin, Jr. and O. Iliopoulos (1998). “The von Hippel-Lindau tumor suppressor protein is required for proper assembly of an extracellular fibronectin matrix.” Mol Cell 1(7): 959-968.
Perez, A., R. M. Rogers and J. H. Dauber (2003). “The prognosis of idiopathic pulmonary fibrosis.” Am J Respir Cell Mol Biol 29(3 Suppl): S19-26.
Piganis, R. A., N. A. De Weerd, J. A. Gould, C. W. Schindler, A. Mansell, S. E. Nicholson and P. J. Hertzog (2011). “Suppressor of cytokine signaling (SOCS) 1 inhibits type I interferon (IFN) signaling via the interferon alpha receptor (IFNAR1)-associated tyrosine kinase Tyk2.” J Biol Chem 286(39): 33811-33818.
Pozzebon, M. E., A. Varadaraj, D. Mattoscio, E. G. Jaffray, C. Miccolo, V. Galimberti, M. Tommasino, R. T. Hay and S. Chiocca (2013). “BC-box protein domain-related mechanism for VHL protein degradation.” Proc Natl Acad Sci USA 110(45): 18168-18173.
Raghu, G., K. K. Brown, U. Costabel, V. Cottin, R. M. du Bois, J. A. Lasky, M. Thomeer, J. P. Utz, R. K. Khandker, L. McDermott and S. Fatenejad (2008). “Treatment of idiopathic pulmonary fibrosis with etanercept: an exploratory, placebo-controlled trial.” Am J Respir Crit Care Med 178(9): 948-955.
Raghu, G., F. J. Martinez, K. K. Brown, U. Costabel, V. Cottin, A. U. Wells, L. Lancaster, K. F. Gibson, T. Haddad, P. Agarwal, M. Mack, B. Dasgupta, I. P. Nnane, S. K. Flavin and E. S. Barnathan (2015). “CC-chemokine ligand 2 inhibition in idiopathic pulmonary fibrosis: a phase 2 trial of carlumab.” Eur Respir J 46(6): 1740-1750.
Raghu, G., D. Weycker, J. Edelsberg, W. Z. Bradford and G. Oster (2006). “Incidence and prevalence of idiopathic pulmonary fibrosis.” Am J Respir Crit Care Med 174(7): 810-816.
Richeldi, L., R. M. du Bois, G. Raghu, A. Azuma, K. K. Brown, U. Costabel, V. Cottin, K. R. Flaherty, D. M. Hansell, Y. Inoue, D. S. Kim, M. Kolb, A. G. Nicholson, P. W. Noble, M. Selman, H. Taniguchi, M. Brun, F. Le Maulf, M. Girard, S. Stowasser, R. Schlenker-Herceg, B. Disse, H. R. Collard and I. T. Investigators (2014). “Efficacy and safety of nintedanib in idiopathic pulmonary fibrosis.” N Engl J Med 370(22): 2071-2082.
To, W. S. and K. S. Midwood (2011). “Plasma and cellular fibronectin: distinct and independent functions during tissue repair.” Fibrogenesis Tissue Repair 4: 21.
Veiling, T., J. Risteli, K. Wennerberg, D. F. Mosher and S. Johansson (2002). “Polymerization of type I and III collagens is dependent on fibronectin and enhanced by integrins alpha 11beta 1 and alpha 2beta 1.” J Biol Chem 277(40): 37377-37381.
Zhou, Q., A. Pardo, M. Konigshoff, O. Eickelberg, G. R. Budinger, K. Thavarajah, C. J. Gottardi, J. Jones, J. Varga, M. Selman, J. I. Sznajder, J. U. Raj and G. Zhou (2011). “Role of von Hippel-Lindau protein in fibroblast proliferation and fibrosis.” FASEB J 25(9): 3032-3044.

What is claimed is:

1. A composition comprising a polypeptide consisting of the following amino acid sequence
(SEQ ID NO: 1)
VRPLQELCRQRIVAAVGRENLARIPLNPVLRDYLSSFPFQI.

2. A composition comprising the peptide in claim 1 or pharmaceutically acceptable salt thereof, and a pharmaceutically acceptable carrier.

3. A composition comprising the peptide in claim 1 in the form of a pharmaceutically acceptable salt wherein said peptide is produced by solid phase peptide synthesis or produced by a yeast cell, mammalian cell or a bacterial cell expression system using one or more nucleic acid encoding the candidate peptide.

4. A polynucleotide comprising nucleotide sequences encoding the peptide in claim 1.

5. A vector comprising the polynucleotide of claim 4.

6. A cell engineered to express the polynucleotide of claim 4.

7. A composition comprising the peptide in claim 1 wherein the composition is a pharmaceutical composition and comprises water and buffer.

8. A pharmaceutical composition comprising the peptide in claim 1 and one or more pharmaceutically acceptable excipients.

9. A method of treating a subject having a disease or conditions characterized by excess matrix deposition or fibrosis, the method comprising administering the composition of claim 1 to a subject.

10. The method of claim 9, wherein the composition is administered intranasally, intrabronchially, intrapleurally or by instillation into the lungs of the subject.

11. The method of claim 9, wherein the fibrosis is pulmonary fibrosis.

12. The method of claim 9, wherein the fibrosis is idiopathic pulmonary fibrosis.

13. The method of claim 9, wherein the composition reduces:
fibronectin matrix or
collagen matrix or
hydroxyproline production of fibroblasts in the subject.

14. The method of claim 9, wherein the composition reduces smooth muscle actin and VHL in the subject.

15. The method of claim 9, wherein the composition reduces fibrosis in the subject.

16. The method of claim 9, wherein the composition restricts formation of the fibronectin matrix in the subject.

17. The method of decreasing the fibronectin and collagen matrix assembly in a subject, comprising administering the composition of claim 1 to the subject.

18. The method of claim 17, wherein the subject has:
an acute lung injury or a fibrotic condition; or
an acute lung injury; or
a fibrotic condition; or
a fibrotic condition comprising pulmonary fibrosis; or
idiopathic pulmonary fibrosis; or
has, or is suspected of having, organ fibrosis.

19. The method of claim 17, wherein the composition is administered by intranasal, intrabronchial or by instillation into the lungs of the subject.

20. The method of claim 17 wherein the pharmaceutical composition is administered as part of a combination therapy regimen.


SEQUENCE LISTING (2 sequences)

SEQ ID NO: 1 - protein, 213 aa, Mus musculus
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKLASGPTSIRVHFQAGRFHLDGSRETFDCLFELLEHYVAAPRRJLLGAPLRQRRVRPLQELCRQRIVAAVGRENLARIPLNPVLRDYLSSFPFQI

SEQ ID NO: 2 - protein, 213 aa, unidentified
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKLASGPTSIRVHFQAGRFHLDGSRETFDCLFELLEHYVAAPRRJLLGAPLRQRRVRPPQELFRQRIVAAVGRENLARIPLNPVLRDYLSSFPFQI